(19) United States
(12) Patent Application Publication
(10) Pub. No.: US 2006/0004524 A1
(43) Pub. Date: Jan. 5, 2006

(54) Method, apparatus, and computer program product for collecting and securing chain of custody information related to a specimen and generated via specimen collection

(75) Inventors: Thomas Gerald Aucoin, Cary, NC (US); John Daniel Ayers JR., Mebane, NC (US); Susan Horne Steltenpohl, Raleigh, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Laboratory Corporation of America Holdings, Burlington, NC (US)

(21) Appl. No.: 11/172,573

(22) Filed: Jun. 30, 2005

Related U.S. Application Data
(60) Provisional application No. 60/584,600, filed on Jul. 1, 2004.
(60) Provisional application No. 60/604,491, filed on Aug. 25, 2004.

(51) Int. Cl. G06F 17/60; G06F 19/00; G01N 33/48; G01N 33/50

(52) U.S. Cl. 702/19; 705/3

(57) ABSTRACT

A specimen collection system, container device, and a method and computer program product for collecting and securing chain of custody (COC) information related to a specimen is provided. The system, method, and computer program product guides a collector to collect a complete COC data set related to a specimen, detect at least one substance in the specimen using a testing device operably engaged with a specimen container configured to receive the specimen, and direct the complete COC data set to an RFID device or bar code label operably engaged with the specimen container.

### CROSS-REFERENCE TO RELATED APPLICATIONS

This application claims the benefit of U.S. Provisional Application No. 60/584,600, filed Jul. 1, 2004, and U.S. Provisional Application No. 60/604,491, filed Aug. 25, 2004, both of which are incorporated herein in their entirety.

### FIELD OF THE INVENTION

The present invention relates generally to collecting and securing a chain of custody data set related to a specimen obtained during specimen collection and/or testing such that the chain of custody data set is completely entered, electronically stored, and made selectively accessible using bar code or RFID technology using, for instance, a printed 2-dimensional (2D) bar code or RFID device operably engaged with a specimen container.

### BACKGROUND OF THE INVENTION

It is common for employers to collect biological specimens including, but not limited to, for example, urine specimens, from existing or prospective employees in order to screen the specimens for substances, such as cocaine, marijuana, and other illegal drugs. In addition, athletic organizations often utilize such specimens to test for banned performance-enhancing drugs and/or hormones. These organizations often employ their own staff or contract employees to collect and pre-test these specimens on-site using, for instance, pre-packaged test strips or test containers, such as the QuickScreen Multi-Panel drug test (Catalog #9178T) available commercially from Phamatech, Inc. Such on-site testing, sometimes called “point-of-collection” testing (POCT), provides a time saving and money-saving pre-screen for the specimens, as only those specimens that result in a “positive” for the target substance(s) need to be tested further at an off-site laboratory. Furthermore, POCT testing can provide a preliminary result in about 5-10 minutes wherein test specimens sent to off-site laboratory facilities may not yield results for 5-10 days. It is also common for documentation and evidentiary purposes to store “positive” POCT specimens at an off-site laboratory or storage facility and it is therefore crucial that chain-of-custody data set related to the specimen is complete as well as properly collected, stored, and secured. In other cases, these organizations simply collect such specimens for later testing at a laboratory (lab-based testing).

Existing methods for collecting and storing chain of custody information during POCT and/or collection and subsequent lab-based testing include the generation of chain of custody documents identifying, for example, the donor, the collector, the time of collection, any collection site identifiers, and (if applicable) preliminary POCT results. These documents are generated at the collection and/or POCT site on paper, and additional COC documents must be produced at a secondary testing/storage site (internal COC forms) for aliquots taken from the from the original specimen. Repeating the COC data entry in this manner is time consuming, labor intensive, and more susceptible to error (either by transposition of data or failure to input important data). In addition, there is no control for ensuring that the COC data set is complete (i.e., the paper forms generated may be missing vital COC data related to the specimen).

Some methods for collecting and storing chain of custody information utilize Radio Frequency Identification (RFID) technology to electronically store chain of custody information, thereby avoiding or minimizing the use of paper COC documents. Such a method is disclosed, for example, in U.S. patent application Publication No. 2003-0183683 to Stewart (“the Stewart application”) which is incorporated by reference herein in its entirety. The disclosed method in the Stewart application, however, may lead to erroneous and/or incomplete COC data entry. For example, the Stewart application does not disclose providing controls for avoiding the incomplete entry of COC data. In addition, bar code labels are sometimes utilized to attach chain of custody or other identifying information related to a specimen to a specimen container in a compact format, thereby avoiding or minimizing the use of paper COC documents. However, conventional bar code printing software may not include prompts for collecting a complete set of COC data or other information that should be collected to complete the COC data set. In addition, conventional COC data collection systems do not include data entry software that guides a specimen collector through approved procedures for properly collecting and documenting a biological sample.

Thus, there exists a need for a collection and/or testing system that provides an intuitive and easy-to-use interface so that an employee may properly collect and/or analyze (in the case of POCT) specimens and be constrained to enter complete and correct COC data into the COC data set. In addition, there exists a need for a specimen collection system associating certain components with the specimen container so as to minimize specimen handling and to closely maintain the COC data with respect to the specimen.

### SUMMARY OF THE INVENTION

The above and other needs are met by the present invention which, in various embodiments, provides a collection system, method, computer program product, and container device for collecting and securing a chain of custody (COC) data set related to a specimen generated during specimen collection for either lab-based testing or POCT, wherein the data within the COC data set is input by a collector and a specimen is provided by a donor. More particularly, a collection system, in one embodiment, comprises: a specimen container for receiving the specimen; an RFID device operably engaged with the specimen container and configured to receive the COC data set; and a computer device capable of communicating with the RFID device. The computer device may be configured to guide the collector to determine data within the COC data set and to guide the collector to input complete data for the COC data set into the computer device. The computer device may also be configured to send the COC data set with complete data to the RFID device for storage therein.

In other embodiments, the specimen container of the collection system may also have a testing device operably engaged therewith for detecting at least one substance in the specimen by directly interacting with the specimen. The specimen container may also have a temperature device operably engaged therewith to determine a temperature of the specimen. In other embodiments, the collection system may further comprise a transceiver in communication with the RFID device and may also process the COC data set with respect to the RFID device.

The method and computer program product embodiments of the present invention are directed to collecting and securing a complete COC data set related to a specimen collected at a specimen collection site, wherein the data within the COC data set is input by a collector and the specimen is provided by a donor. In one embodiment, the method comprises the following steps: guiding the collector, via a computer device, to determine the data within the COC data set and to input the complete data for the COC data set in to the computer device, and directing the COC data set with complete data to an RFID device, wherein the RFID device is operable engaged with a specimen container configured to receive the specimen.

In other embodiments, the method and computer program product embodiments of the present invention may comprise additional steps of: detecting at least one substance in the specimen using a testing device operably engaged with the specimen container so as to be capable of directly interacting with the specimen; and inputting a result provided by the testing device into the computer device, wherein the result is included in the COC data set. In other embodiments, wherein the COC data set is to be transferred to a bar code label (such as a 2D bar code label), the method and computer program product embodiments may further comprise directing the COC data set to a printer operably engaged with the computer device, wherein the printer may be configured to print the COC data set as a bar code label configured to be capable of operably engaging a specimen container configured to receive the specimen.

The container device embodiments of the present invention provide a container device for receiving a specimen collected during specimen collection and/or testing where the specimen is provided by a donor. More particularly, the container device comprises a testing device configured to detect at least one substance in the specimen, the testing device being capable of directly interacting with the specimen.

In other embodiments, the container device of the present invention may also comprise an RFID device configured to receive a COC data set that is related to the specimen, wherein the data with the COC data set is input by a collector. In some advantageous embodiments of the container device, the RFID device may be further configured to direct a result provided by the testing device to the RFID device for retention therein. The container device may also further comprise a temperature device or tamper-evident device operably engaged therewith.

Thus, embodiments of the present invention guide the collector to determine data within a COC data set, and to input complete data into the COC data set via a computer device at a specimen collection site such that the data set may be printed or otherwise transferred to a bar code label (such as a 2D bar code label) or other device that may be operably engaged with a specimen container. In some embodiments, a specimen container is provided with which an RFID device is operably engaged such that there is a reduced risk of mislabeling a specimen container with incomplete and/or inaccurate data within the COC data set. In addition, some advantageous embodiments include a specimen testing device and/or a temperature device so as to provide on-site (POCT) test capabilities that require no additional steps on the part of the collecting technician, such as recording the lot number and expiration date of a testing device or temperature device that is separate from the specimen container. The testing device also provides that preliminary POCT test results remain associated with the corresponding specimen.

Embodiments of the present invention provide these and other advantages as further discussed herein.

### BRIEF DESCRIPTION OF THE DRAWINGS

Having thus described the invention in general terms, reference will now be made to the accompanying drawings, which are not necessarily drawn to scale, and wherein:

FIG. 1 is a schematic of one embodiment of the specimen collection and/or testing system of the present invention;

FIG. 2 is a schematic representation of a chain of custody data input screen according to one embodiment of the present invention, depicting categories of chain of custody data that may be collected during the course of specimen collection and/or POCT;

FIG. 3 is a flowchart illustrating the various steps of the specimen collection and/or testing method for collecting and securing chain of custody data according to one embodiment the present invention;

FIG. 4 is a flowchart illustrating various steps for operating a specimen collection and/or testing system including guiding a collector to determine and input chain of custody data, receiving chain of custody data, and directing the chain of custody data set to the RFID device, according to one embodiment of the present invention;

FIG. 5 is a flowchart illustrating various steps for operating a specimen collection and/or testing system including guiding a collector to determine and input chain of custody data, receiving chain of custody data, directing the chain of custody data set to the RFID device, directing a collector (using a testing device) to detect a substance in the specimen, and directing a result provided by the testing device to the RFID device, according to one embodiment of the present invention;

FIG. 6 is a flowchart illustrating various steps for operating a specimen collection and/or testing system including guiding a collector to determine and input chain of custody data, receiving chain of custody data, directing the chain of custody data set to the RFID device, directing a collector (using a testing device) to detect a substance in the specimen, directing the result provided by the testing device to the RFID device, receiving an electronic signature from a donor, and receiving an electronic signature from a collector, according to one embodiment of the present invention;

FIG. 7 is a schematic of an input screen according to one embodiment of the present invention illustrating the collector being guided to input chain of custody data related to the lot number and expiration date of the testing device used in POCT testing;

FIG. 8 is a schematic of an input screen according to one embodiment of the present invention illustrating the collector being guided in the collection of a specimen from a donor according to a standard operating procedure;

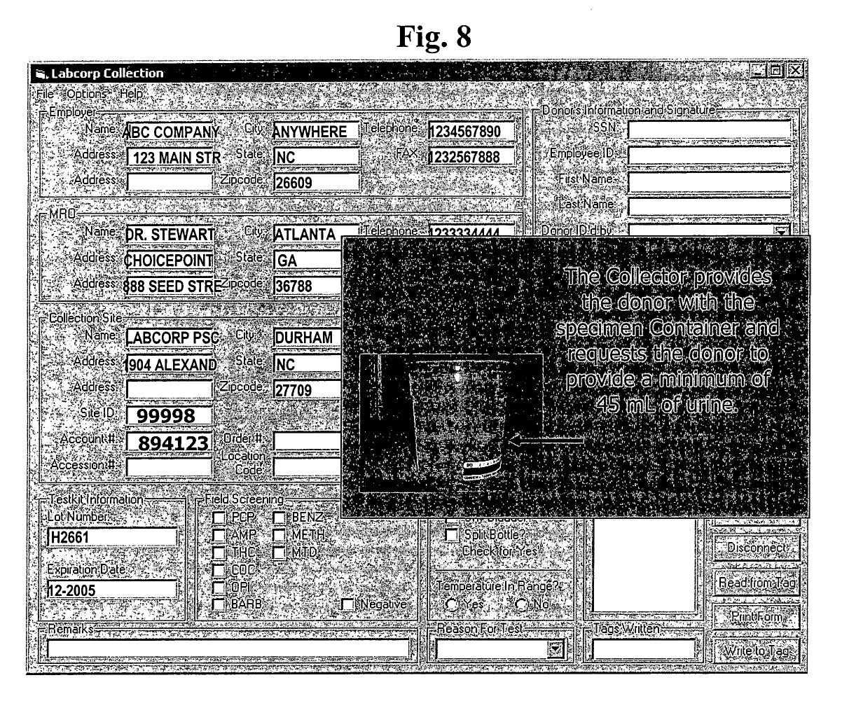

FIG. 9 is a schematic of an input screen according to one embodiment of the present invention illustrating the collector being directed to read a result provided by a testing device interacting with the specimen;

FIG. 10 is a schematic of an input screen according to one embodiment of the present invention illustrating the collector being guided to input chain of custody data in a specimen collection;

FIG. 11 is a flowchart illustrating the various steps of the specimen collection and/or testing method for collecting and securing chain of custody data to be printed on a bar code label according to one embodiment the present invention;

FIG. 12 is a flowchart illustrating various steps for operating a specimen collection and/or testing system including guiding a collector to determine and input chain of custody data, receiving chain of custody data, and converting the chain of custody data set to bar code data, according to one embodiment of the present invention;

FIG. 13 is a flowchart illustrating various steps for operating a specimen collection and/or testing system including guiding a collector to determine and input chain of custody data, receiving chain of custody data, converting the chain of custody data set to bar code data, directing a collector (using a testing device) to detect a substance in the specimen, and directing a result provided by the testing device to a computer device for conversion to bar code data, according to one embodiment of the present invention; and

FIG. 14 is a flowchart illustrating various steps for operating a specimen collection and/or testing system including guiding a collector to determine and input chain of custody data, receiving chain of custody data, converting the chain of custody data set to bar code data, directing a collector (using a testing device) to detect a substance in the specimen, directing the result provided by the testing device to a computer device for conversion to bar code data, receiving an electronic signature from a donor, and receiving an electronic signature from a collector, according to one embodiment of the present invention.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Embodiments of the present invention are described herein in conjunction with the collection of specimens for the subsequent detection of the existence of commonly-abused drugs. One skilled in the art will appreciate, however, that the embodiments of the present invention can be utilized in a variety of other applications, including the collection and testing of various types of biological specimens that may be collected for lab-based testing and/or tested via POCT technologies, such as, for instance urine samples, blood samples, cheek swabs, sweat samples or other biological fluid samples. In addition, the embodiments of the present invention may be utilized in the collection of specimens such as hair specimens for use in criminal investigations or forensic investigations. In addition, embodiments of the present invention are discussed with respect to chain of custody (COC) data related to collected specimens, for instance, to show that proper COC protocols were met in the handling of evidence by police investigators, coroners, medical personnel, and other authorities involved in criminal investigations and/or specimen collection and testing. Thus, one skilled in the art will appreciate that the exemplary embodiments disclosed an described herein are not intended to be limiting in any manner with respect to the present invention.

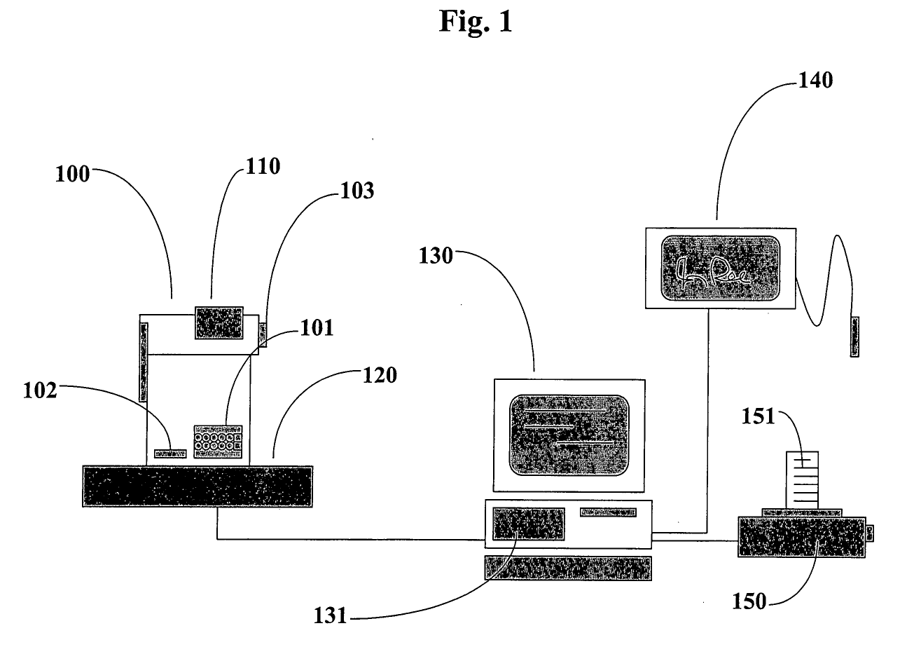

FIG. 1 shows one embodiment of a collection system according to the present invention which comprises: a specimen container 100, an RFID unit 110, a transceiver 120 configured to read and/or write data to the RFID unit 110, and a computer device 130 configured to receive data, such as, for instance chain of custody data related to a specimen. The RFID unit 110 may be operably engaged with the specimen container 100 or in other embodiments, may be selectively attachable to or non-removably engaged with the specimen container 100. According to other advantageous embodiments, the collection system may include a computer device 130 capable of communicating with the RFID unit 110 without the use of a separate transceiver 120.

In other advantageous embodiments, the specimen container 100 may also be provided with a testing device 101 capable of detecting one or more substances in the specimen. The testing device 101 may be provided separate from the specimen container, and exposed to the specimen contained therein to perform a point-of-collection (POCT) test of the specimen. Testing devices suitable for interacting with a specimen contained in a specimen container include, for instance the QuickScreen Catalog # 9178T from Phamatech, Inc. which provides a testing device 101 on a card form having a plurality of windows defined therein. The windows define color-change reactive test areas that visually indicate the presence of substances in the specimen upon the physical interaction of the test areas with the specimen, such as, by dipping the card into a liquid specimen. In some embodiments, the testing device 101 may further comprise test sticks configured to be dipped into a liquid biological specimen. In other embodiments, the testing device 101 may be further configured to interact with other types of biological samples, both liquid and solid, in order to visually indicate the presence of substances in the specimen. For instance, the testing device 101 may be configured to interact with hair samples, sweat samples, saliva samples, blood samples, and/or other types of biological specimens.

In other embodiments, the POCT testing device 101 may be disposed in or otherwise engaged with, for instance, the inner surfaces of the specimen container 100. Such testing devices are integrated with a specimen container in some specimen containers having no RFID unit or other data collection mechanism such as in QuickScreen Catalog #9177x and #9178x from Phamatech, Inc. In such embodiments, the surfaces of the specimen container and or the testing device may define apertures, or include other mechanisms for providing interactive communication directly between the testing device and the specimen. In other embodiments, the testing device 101 may be operably engaged with the interior or other surface of the specimen container so that the testing device 101 may directly interact with the specimen contained therein. In some embodiments of the collection system, and specimen container that include the testing device 101, the testing device 101 may include an indicator mechanism using, for example, symbols and/or color changes that become visible on the surface of the testing device 101, or through the specimen container 100 (which may be composed of translucent and/or transparent material) to indicate the presence of one or more substances in the specimen as detected by the testing device 101. The testing device 101 may be configured to detect, for instance, drugs such as PCP, amphetamines, methamphetamines, methadone, THC (THCA), THC metabolites, cocaine (benzoyl-ecgonine), cocaine metabolites, oxycodone, morphine, ecstasy, propoxyphene, opiates, methaqualone, benzodiazopenes, other narcotics, and combinations thereof. One skilled in the art will appreciate that the testing device 101 may also be configured to include, for example, an assay and/or a test strip suitable for detecting and indicating the presence of any of a variety of substances that may be present in a specimen. According to some embodiments, the testing device 101 may be an electronic device configured to detect and indicate the presence of substances within the specimen. In embodiments where the testing device 101 is an electronic device, the testing device may be further configured to communicate with the RFID device 110 and/or the computer device 130 of the present invention to provide a test result as part of the data in the complete COC data set.

In yet another advantageous embodiment, the specimen container 100 may be provided with a temperature device 102 operably engaged with the specimen container 100, that is configured to determine and/or display the temperature of the specimen. The temperature device 102 may indicate temperature ranges by, for instance, a color-coded strip visible to the collector on the exterior of the specimen container. In some embodiments the temperature device may also comprise a mercury thermometer having a Celsius or Fahrenheit numerical scale. In yet another embodiment, the temperature device 102 may be an electronic device, such as a thermistor, that may be digitally coupled to the RFID device 110 such that the temperature data history of the specimen may be stored as part of the COC data set.

In other advantageous embodiments, the specimen container 100 may also be provided with a tamper-evident device 103 configured to seal and/or provide tamper-evident information in case the specimen container 100 is opened after the tamper-evident device has been affixed thereto. The tamper evident device 103 may comprise, for example, an adhesive sticker, tape, or another appropriate tamper-evident mechanism. As illustrated in FIG. 1, a collection system according to one embodiment of the present invention may include a transceiver 120 configured to process the COC data set with respect to the RFID device 110 operably engaged with the specimen container 100. The transceiver 120 may be connected via a wire or wireless connection, to a computer device 130, such as, for instance, a personal computer, PDA, or other processing unit suitable for receiving and processing data related to the specimen. As such, the computer device 130 may be used to input COC data related to the specimen, the donor, the collector, and the testing device 110 as well as the result of a POCT test to detect one or more substances in the specimen. The computer device 130 may also be configured to store the COC data set in a memory 131 and/or selectively direct the COC data set to the RFID device 110 operably engaged with the specimen container 100. In addition, the computer device 130 may be configured to include a password-protected user-interface, such that the computer device 130 cannot be accessed by unauthorized personnel. The transceiver 120 and computer device 130 are also capable of receiving a COC data set from the RFID device 110 operably engaged with the specimen container 100, such that a user of the collection system may access and process the data within the COC data set contained in the RFID device and/or copy the COC data set contained in the RFID device. Thus, the transceiver 120 and computer device 130 are capable of copying a COC data set from a first RFID device operably engaged with a first specimen container containing a given specimen, storing the data in memory 131, and transferring the COC data set to a second RFID device operably engaged with a second specimen container in cases where, for instance, the given specimen requires separation into two separate specimen containers, such as aliquot tubes used to confirm POCT tests at a secondary testing site.

A collection system in one embodiment of the present invention may also include an electronic pad 140, or other appropriate device, configured to capture electronic signatures of the donor and/or collector and transfer this information electronically to the computer device 130 for storage in the memory 131, and/or transfer the information to an RFID device 110 via a transceiver 120. In addition, a collection system in one embodiment of the present invention may also include a printer device 150 configured to be capable of printing a bar code label 151 coded with the COC data set such that the bar code label 151 may be affixed to a surface of the specimen container 100.

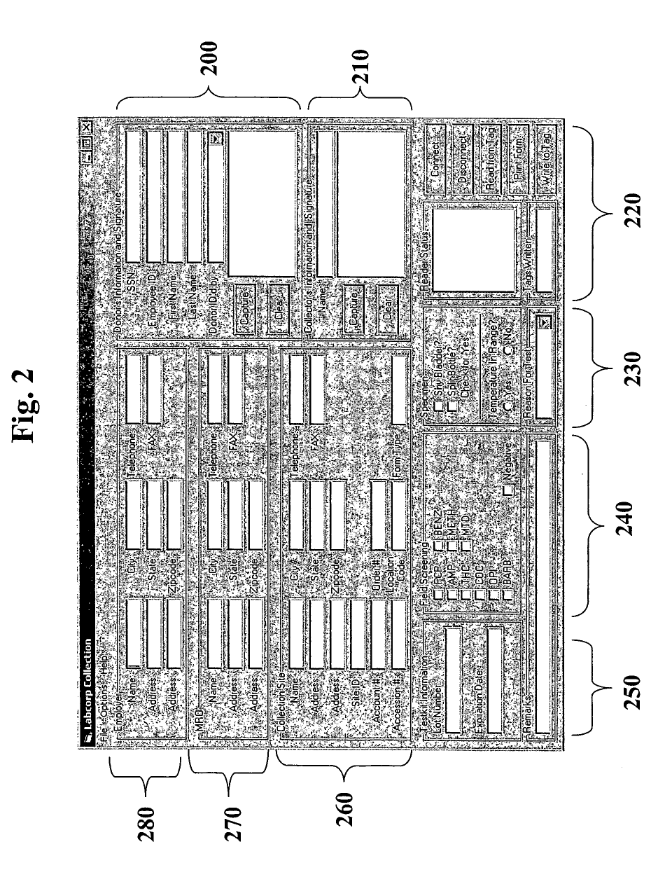

FIG. 2 depicts an input screen for a COC data set with respect to the computer device 130 executing an associated computer program product according to one embodiment of the present invention. A user interface showing the COC data contained within a COC data set is also provided. In some embodiments, such as that depicted in the input screen of FIG. 2, the collector may be guided to completely input data associated with a complete COC data set. For instance, the input screen may include a donor identification field 200 for receiving donor identification information, such as, for example, the donor's Social Security number or employee number, first and last name, and the method by which the collector at the POCT site identified the donor. This feature of the present invention is necessary in POCT testing environments where the collector is required to identify the donor providing the specimen, either by photo identification, or by sight if the collector knows name and likeness of the donor. The donor identification field 200 may also require an electronic signature from the donor, that may be captured, for instance, on an electronic pad 140 in communication with the computer device 130.

The input screen also may include a collector identification field 210, including the collector's name and electronic signature, which may be captured electronically on an electronic pad 140 in communication with the computer device 130. In addition, the input screen may also include transceiver (“reader”) status field 220 which may also include controls (such as, for instance, virtual “buttons”) for processing COC data with respect to the transceiver 120 and RFID device 110. Such processing may include, for instance, sending a complete COC data set to an RFID device 110 or reading a complete COC data set from an RFID device 110.

The input screen may also include specimen information data 230, wherein the collector may indicate whether the specimen was divided into two separate specimen containers 100 and whether the temperature of, for instance, a urine specimen, as indicated by a temperature device 102, is within the range specified for the specimen by applicable urine specimen guidelines. In addition, in the case of urine specimens, the specimen information field 230 may include notations regarding anomalies in collecting the specimen. For instance, if the donor experiences “shy bladder” the collector may make this notation in the specimen information field 230 of the input screen.

The data input screen depicted in FIG. 2 may also require preliminary POCT test result data, via a field screening data field 240, where the collector may input and store a result of a preliminary screening, as detected during POCT screening of the specimen, using, for instance, the testing device 101 described in conjunction with FIG. 1. The field screening data field 240 may also include a check box for a negative POCT result (where none of the substances being tested for are present in the specimen). The POCT result entered into the field screening data field 240 may then be transferred to the RFID device 110 via, for instance, the transceiver 120, so that the POCT result data are included as part of the COC data set related to the specimen. Thus, if the specimen is shipped to a second laboratory site for further screening and/or storage, a second transceiver 120 and associated computer device 130 located at the second laboratory site may be used to read the complete COC data set (including POCT results) so as to determine if further confirmation testing is needed (for example, in cases where the field screening data field 240 indicates a “positive” result for any of the target substances).

Data from the testing device 101 may also be included as part of the input screen in a test kit information field 250. The collector may be required to input, for example, the identifying lot number and expiration date of the testing device 101 used to perform POCT tests on the specimen. In embodiments of the present invention where the testing device 101 is operably engaged with the specimen container 100, the collector may also record the lot number and expiration date of the specimen container/testing device apparatus in the test kit information field 250.

The input screen depicted in FIG. 2 may also include a collection site data field 260, a Medical Review Officer (MRO) data field 270, and an employer information data field 280 (for embodiments of the present invention used to collect specimens from current or prospective employees of that employer). This information may include, for example, addresses; site codes; names of employer, MRO, and/or collection site; and phone and fax numbers for the employer, MRO, and/or collection site, as shown in FIG. 2.

FIGS. 1, 3, and 11 illustrate a method for collecting and securing chain of custody data for a specimen collected during point-of-collection testing according to one embodiment of the present invention. First, in step 300, the collection system guides the collector to collect a complete set of COC data, and to input the data into the computer device 130. The collector may input the COC data via keystrokes on a keyboard in communication with the computer device 130 such that the data fields of the input screen of FIG. 2 are completed. In other embodiments, the collector may input COC data via a touch-screen monitor or other input device in communication with the computer device 130. Step 300 may include the collection of the donor's electronic signature (Step 301), the collector's electronic signature (Step 302), and /or the POCT Screening result (Step 303) obtained in the optional detecting step 310. One skilled in the art will appreciate that COC data in the data set may vary depending on the purpose of collecting the specimen as well as the standard operating procedures of the collection and/or testing site. One skilled in the art will also appreciate that the required fields of the COC data set (depicted in FIG. 2) and the computer program product of the present invention (described below) may be modified to guide the collector to properly input a complete COC data set as depicted in step 304. This may be accomplished, for instance, by preventing the collector from directing an incomplete COC data set to the RFID device. In some cases, such as urine specimen screening for drugs as requested by an employer, the complete COC data set will include data substantially similar to the data depicted in FIG. 2. In this case, the COC data set may include the collection of data specific to POCT testing of urine specimens, such as data related to minimum specimen amounts 305 and whether the donor exhibited “shy bladder” 306 that interfered with the POCT testing procedure.

In some advantageous embodiments of the present invention, step 310 is performed, wherein the collector determines whether one or more substances are present in the specimen using, for example a testing device 101 that may be operably engaged with the specimen container 100, as previously discussed.

Thereafter, in some embodiments of the present invention, step 320 may also be performed, wherein the collector inputs the results of the detecting step 310 as COC data to form a complete COC data set. In one embodiment, the results of the detecting step 310 may be entered in the “field screening” data field 240 depicted in the input screen depicted in FIG. 2.

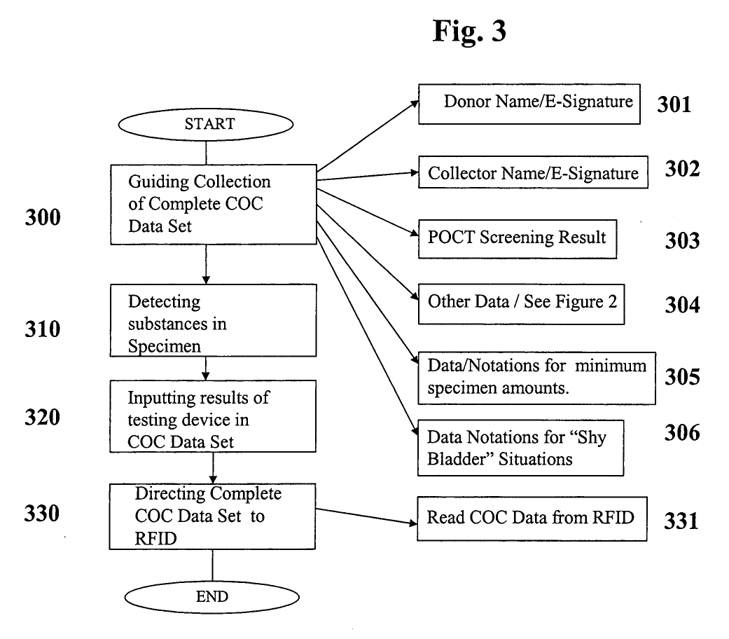

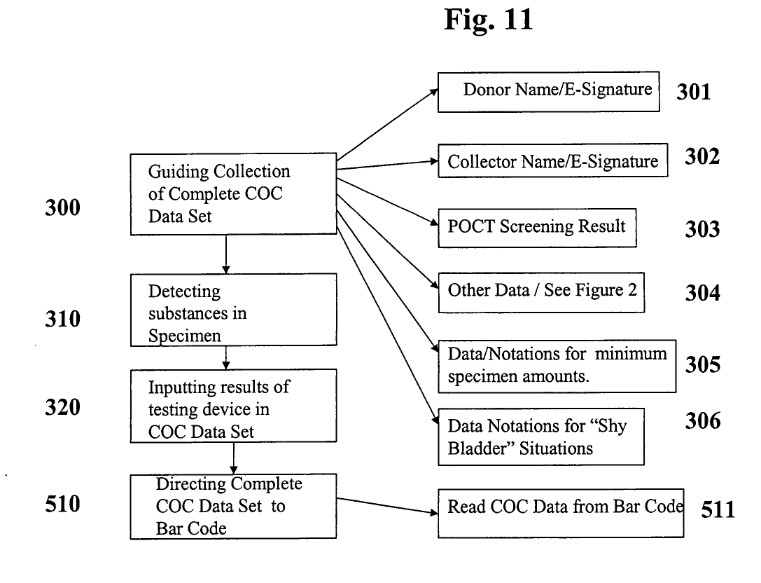

In step 330 the collector directs the complete COC data set, collected and entered during step 300 and optionally during step 320, to the RFID device 100 via, for instance a transceiver 120. This step may be accomplished using, for instance, the reader status field 220 shown in FIG. 2 by clicking on a button in the reader status section labeled “Write to Tag” as shown in the reader status field 220. In addition, the collector at this step may also use the input screen to read data from the RFID device 110 via the transceiver 120 by clicking on the “Read from Tag” button in the reader status field 220 as illustrated schematically in step 331 of FIG. 3. According to other method embodiments, (as illustrated schematically in FIG. 11), in step 510 the collector may direct the complete COC data set from the computer device 130 to a printer 150 configured to print the COC data set as a bar code label 151 that may then be affixed, for instance, to the specimen container 100. The printer 151 (and computer device 130 operably engaged therewith) may be configured to convert the COC data set into a bar code label 151 wherein the bar code printed on the label is in a number of different bar code formats which may include, but are not limited to: two-dimensional (2D) bar code; code 128; PDF-417; data matrix; or other bar code formats suitable for encoding the complete COC data set. In addition, FIG. 11 further illustrates optional step 511 wherein the computer device 130 or other device (such as, for instance, a laser scanner or other bar code reader) may be used to read the complete set of COC data from the bar code label 151 that may be affixed to the specimen container 100 such that a clinician or other personnel may receive a body of text corresponding to the complete set of COC data.

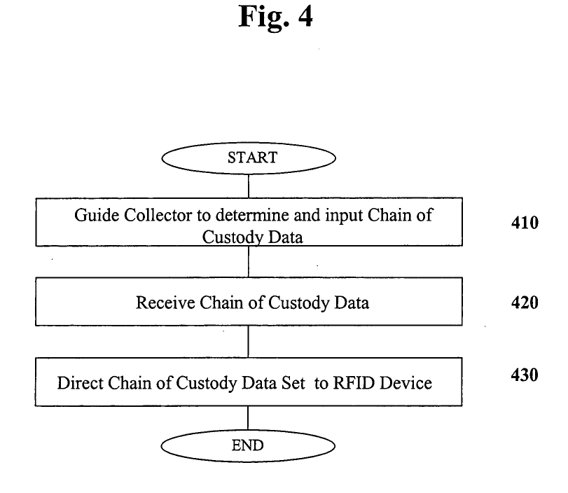

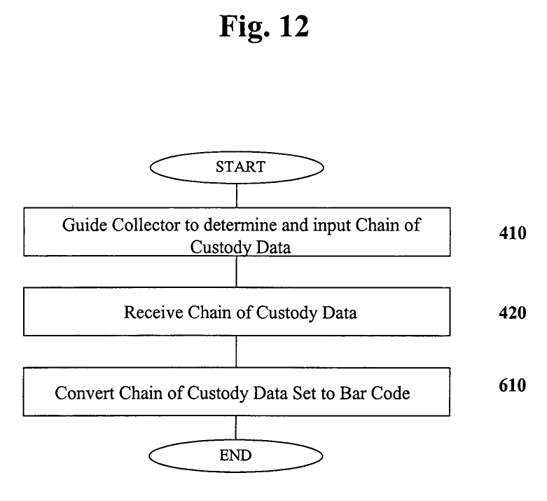

According to one embodiment of the present invention, as illustrated in FIG. 4, the computer device 130, executing an appropriate computer program product, implements a method for collecting and securing chain of custody data for a specimen whereby, in block 410, a processing portion is configured to guide the collector to determine and input chain of custody (COC) data into the computer device 130. In block 420, another processing portion is configured to receive the COC data, and in block 430, another processing portion is configured to direct the completed COC data set to the RFID device 110 via, in some instances, a transceiver 120. In an alternate embodiment, the computer device 130, executing an appropriate computer program product, may (as shown in FIG. 12) execute alternate step 610 wherein a processing portion may be configured to convert the completed COC data set to a bar code format such that the COC data set may be printed on a bar code label 151 (in, for instance, two-dimensional (2D) bar code) that may be printed by a printer 151 operably engaged with the computer device 130. In addition, the bar code label 151 may then be affixed to a surface of a specimen container 100 so as to attach the COC data set information directly to the pertinent specimen. Thus, the bar code label 151 may then be interrogated by a laser scanner or other bar code reader device such that the COC data set may be recovered from the bar code label when the specimen container 100 is received by, for instance, an off-site processing or testing lab (as depicted in FIG. 11, step 511).

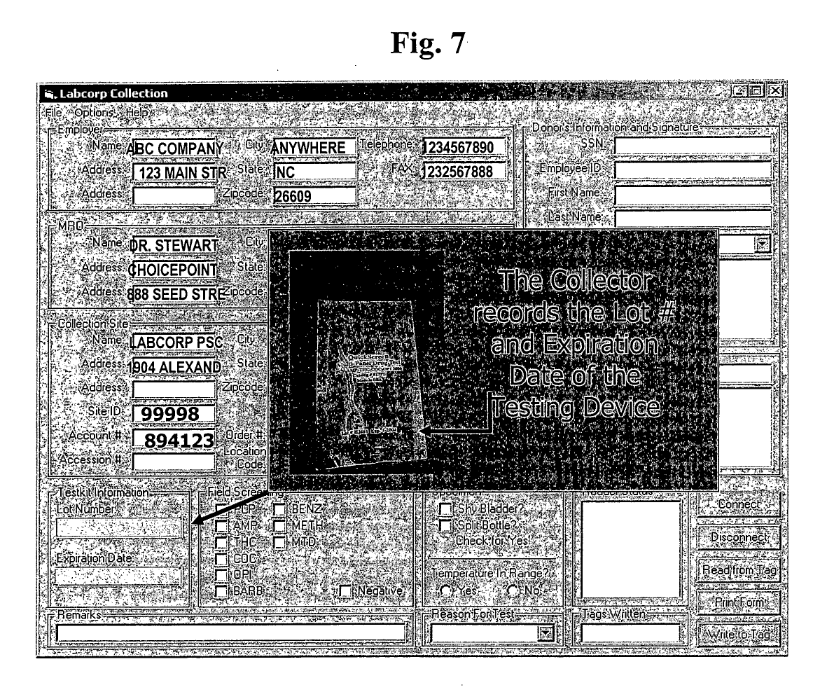

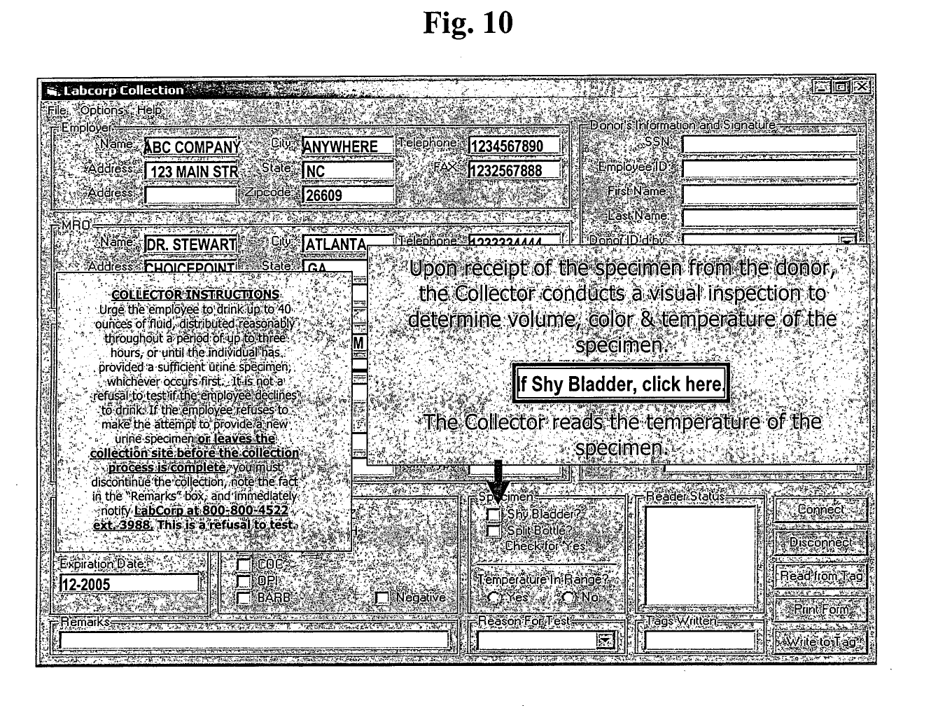

The processing portion for guiding, depicted in block 410, may also be configured, for instance, to direct the computer device to provide an on-screen guide to the collector to direct the collector to enter lot number and expiration data for the testing device 101 used to detect a substance in the specimen. An example of such a guide for the collecting of COC data is depicted in FIG. 7. The processing portion for receiving, of block 420, may be further configured to direct reception of COC data input via computer keystrokes on a keyboard, clicks of a mouse on virtual control “buttons” that are produced on the input screen, such as that depicted in FIG. 2, or via other appropriate user-interface techniques, such as, for example, voice recognition software coupled to a microphone. The processing portion for guiding of block 410 may also be configured to provide visual and textual prompts for guiding the collector, for example, on the amount of a specimen that should be collected. Such an instructional guide is illustrated in the context of collection of a urine specimen in FIG. 10. The processing portion for guiding of block 410 may also be configured to guide the collector in dealing with and recording data related to, for example, a donor's “shy bladder,” wherein the donor may be unable to provide a urine specimen in the presence of the collector. (The presence of the collector is typically required by specimen collection procedures to ensure the validity of tests on the urine specimen). In this case, the processing portion for guiding of block 410 may also be configured to provide an instructional window on the input screen in response to the collector checking a “shy bladder” box in the specimen data 230 of the input screen. An example of “shy bladder” instructions for the collector is shown in the instructional window of the present invention in FIG. 12. Finally, the processing portion for directing of block 430 may be configured to transmit COC data from, for instance, the memory 131 of the computer device 130, via a wire or wireless connection (such as, for instance, IR, RF, or other wireless connection) an to an RFID device 110 operably engaged with the specimen container 100 as previously described. In some instances, the COC data may be directed from the memory 131 to the RFID device 110 via a transceiver 120. In other instances, the COC data may be transferred from the memory 131 of the computer device 130, via a wire or wireless connection (such as, for instance, IR, RF, or other wireless connection) an to a printer 150 configured to print the COC data set in a bar code format on a bar code label 151 that may be affixed to a surface of the specimen container 100.

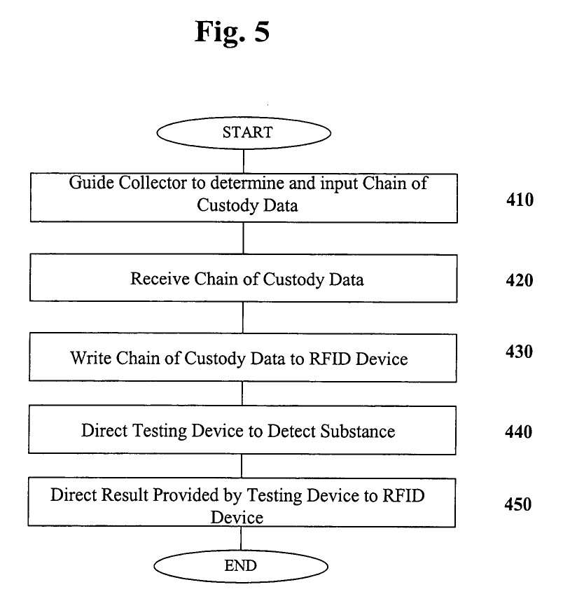

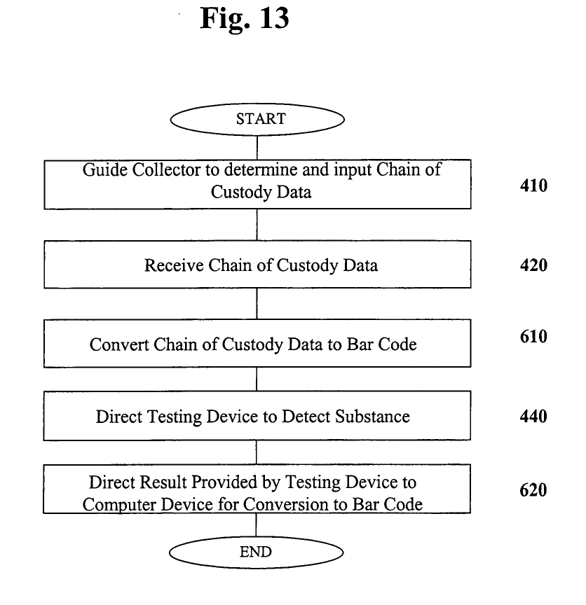

As illustrated in, FIG. 5 and FIG. 13, the computer device 130, executing an appropriate computer program product, implements a method for collecting and securing chain of custody data for a specimen whereby, the processing portions of blocks 410, 420, and 430 (or alternatively, block 610) configured to guide the collector, receive the COC data, and direct the completed COC data to the RFID device or to a printer 150 configured to produce a bar code label 151, as previously discussed, precede the processing portions of blocks 440 and 450 respectively. The processing portion of block 440 is configured to direct the testing device 101 to perform a POCT test to: detect to the presence of a substance in the specimen, determine the result of the test, and input the test result into, for example, the field screening data field 240, shown in FIG. 2 such that the test result is stored in the memory 131 of the computer device 130. The processing portion of block 440 is compatible with embodiments of the present invention wherein the testing device 101 includes, for instance, electronic components configured to electronically determine the present of substances in the specimen as well as electronic components configured to communicate directly with the computer device 130 and/or RFID device 110. As shown in block 450, another processing portion is configured to direct the computer device 130 to send the test result (as part of, for example, the complete COC data set) to the RFID device 110 for storage therein. As discussed above, in some instances the COC data may be directed from the memory 131 to the RFID device 110 via a transceiver 120. Alternatively, as shown in FIG. 13, a processing portion shown in block 620 may direct the electronic test results for substances within the specimen to the computer device 130 and/or an attached printer 150 such that the test results may be included with the COC data set and the resulting bar code label 151 that may be printed to contain the COC data set.

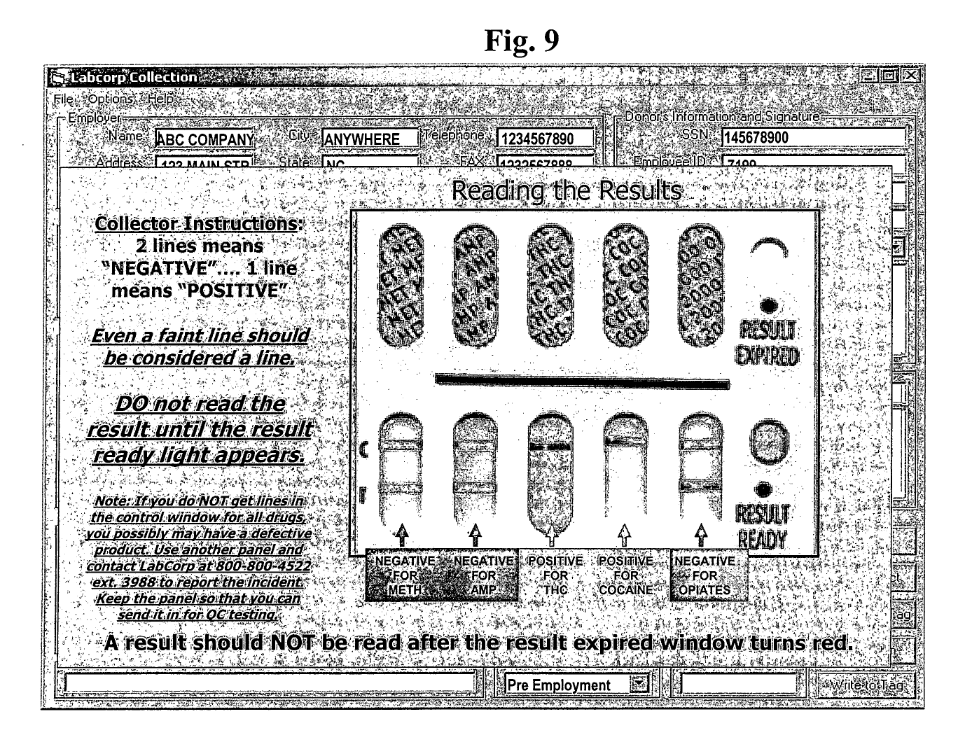

The processing portion for directing, of step 440, may further be configured to direct the computer device 130 to display as part of the input screen (such as that shown in FIG. 2) the field screening data field 240. In this embodiment, the collector performs a preliminary drug test on a urine specimen by exposing a test kit strip, such as for example, a test strip Catalog # 9178T provided by PhamaTech, Inc. to a specimen to detect the presence of one or more substances that may be present in the specimen. The collector may then read the preliminary results of the test as directed by the computer program product of the present invention. For example, the processing portion for directing, in block 440, may be configured to produce an instruction screen such as that shown in FIG. 9 instructing the collector on how to properly read a result from the testing device 101 being used at the testing site. Furthermore, processing portion for directing, in block 450, may be further configured to direct the test result (such as, for instance, positive test results for drugs detected in a urine specimen) by determining the appropriately checked boxes depicted in the field screening data field 240 depicted on the input screen of FIG. 2. The processing portion for directing of block 450 may further be configured to direct the test result to the RFID device 110. In other embodiments, as shown in FIG. 13, block 620 shows a processing portion for directing the test result to the computer device 130 such that the test result may be passed on to a printer device 150 operably engaged with the computer device 130 and configured to be capable of printing a bar code label 151 suitable for affixing to a surface of the specimen container 100.

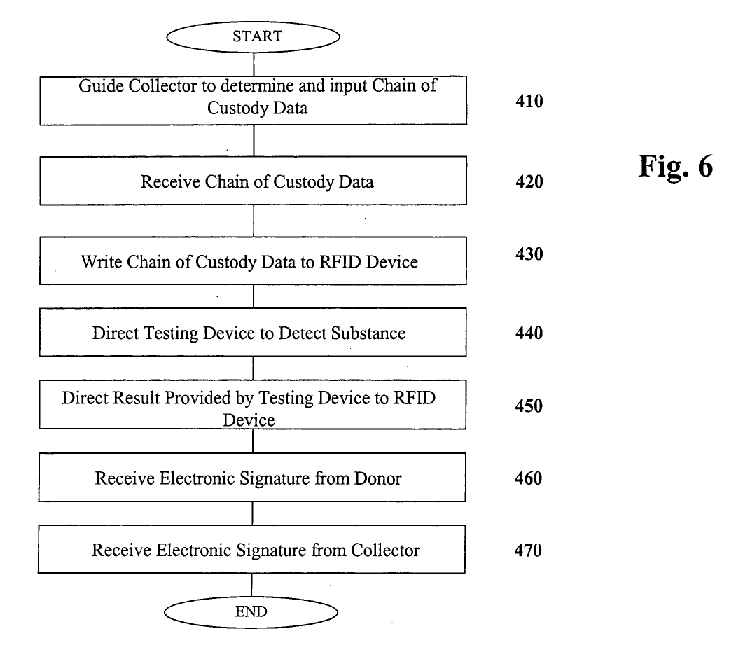

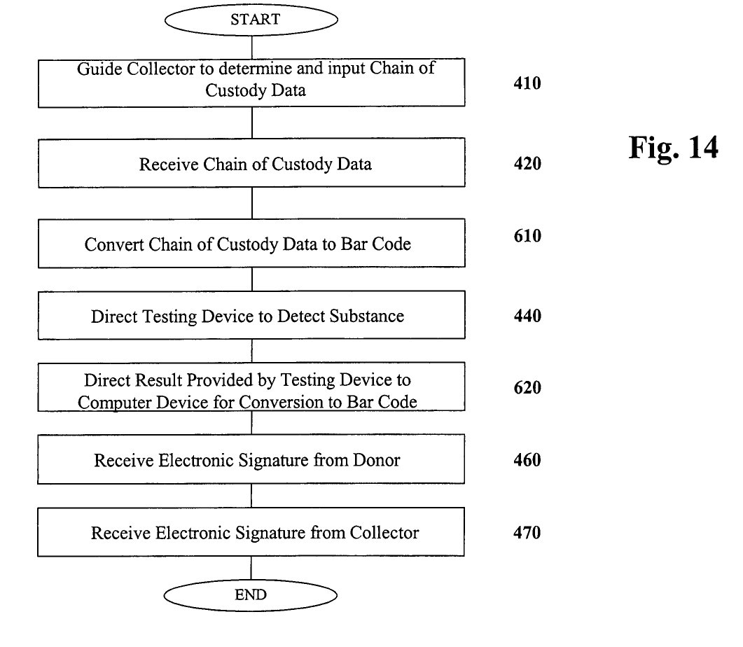

In accordance with the method and computer program product flowchart depicted in FIGS. 6 and 14, the computer program product of the present invention may further include executable portions for directing the computer device 130 to receive an electronic signature from a donor and a collector. Block 460 depicts the receiving of a donor's electronic signature, and block 470 depicts the receiving of a collector's electronic signature. For instance, the processing portions for receiving of blocks 460 and 470 may be configured to display an input screen such as that shown in FIG. 2, including a donor's information field 200 and a collector's information field 210. Furthermore, the computer device 130 may be further connected to an electronic pad 140, the electronic pad 140 configured to receive a written signature provided by the donor and/or collector using, for instance, a stylus configured to digitize a written signature. Furthermore, the collector may click on the “Capture” button depicted in donor and collector information fields 200 and 210, respectively, of FIG. 2 in order to transfer the electronic signatures from the electronic pad 140 to the memory 131 of the computer device 130.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A collection system adapted to collect and secure a chain of custody data set related to a specimen collected during specimen collection, data within said chain of custody data set being adapted to be input by a collector and said specimen being adapted to be provided by a donor, said collection system comprising:
a specimen container for receiving said specimen;
an RFID device operably engaged with said specimen container, said RFID device being configured to receive said chain of custody data set;
a computer device capable of communicating with said RFID device and configured to guide said collector to determine said data within said chain of custody data set and to input complete data for said chain of custody data set into said computer device, said computer device also being configured to send said chain of custody data set with complete data to said RFID device for retention therein.

2. A collection system according to claim 1, further comprising a transceiver in communication with said RFID device and configured to process said chain of custody data set with respect to said RFID device.

3. A collection system according to claim 1, further comprising a testing device, said testing device being operably engaged with said specimen container so as to be capable of directly interacting with said specimen and said testing device being configured to detect at least one substance in said specimen.

4. A collection system according to claim 3, wherein said testing device is configured to detect a substance selected from the group consisting of: PCP;
amphetamines;
methamphetamines;
methadone;
benzodiazopenes;
barbiturates;
THC;
THC metabolites;
THCA;
cocaine;
cocaine metabolites;
ecstasy;
oxycodone;
propoxyphene;
methaqualone
opiates; and
combinations thereof.

5. A collection system according to claim 1, further comprising a temperature device operably engaged with said specimen container, said temperature device being configured to determine a temperature of said specimen.

6. A collection system according to claim 1, wherein said RFID device is non-removably engaged with said specimen container.

7. A collection system according to claim 1, further comprising a tamper-evident device operably engaged with said specimen container.

8. A method for collecting and securing a chain of custody data set related to a specimen collected during specimen collection, data within said chain of custody data set being adapted to be input by a collector and said specimen being adapted to be provided by a donor, said method comprising the steps of:
guiding said collector, via a computer device, to determine said data within said chain of custody data set and to input complete data for said chain of custody data set into said computer device; and
directing said chain of custody data set with complete data to an RFID device, said RFID device being operably engaged with a specimen container configured to receive said specimen.

9. A method according to claim 8, further comprising detecting at least one substance in said specimen with a testing device operably engaged with said specimen container so as to be capable of directly interacting with said specimen.

10. A method according to claim 9, further comprising inputting a result provided by said testing device into said computer device, said result being included in said chain of custody data set.

11. A computer program product capable of directing the collection and securement of a chain of custody data set related to a specimen, data within said chain of custody data set being adapted to be input by a collector and said specimen being adapted to be provided by a donor, the computer program product comprising a computer-readable storage medium having computer-readable program code portions stored therein, the computer-readable program code portions comprising:
an executable portion for guiding said collector to determine data within said chain of custody data set and to input complete data for said chain of custody data set into a computer device; and
an executable portion for directing said chain of custody data set to a transceiver configured to direct said chain of custody data set to an RFID device operably engaged with a specimen container configured to receive said specimen.

12. A computer program product according to claim 11, further comprising:
an executable portion for directing a testing device to detect at least one substance in said specimen, said testing device being operably engaged with said specimen container so as to be capable of directly interacting with said specimen received therein; and
an executable portion for directing a result provided by said testing device to an RFID device operably engaged with said specimen container.

13. A computer program product according to claim 11, further comprising:
an executable portion for receiving data selected from the group consisting of: an electronic signature from said donor, an electronic signature from said collector, and combinations thereof.

14. A container device adapted to receive a specimen collected during point-of-collection testing, said specimen being adapted to be provided by a donor, said container device comprising a testing device configured to detect at least one substance in said specimen, said testing device being capable of directly interacting with said specimen received therein.

15. A container device according to claim 14, further comprising an RFID device configured to receive a chain of custody data set related to said specimen, data within said chain of custody data set being adapted to be input by a collector.

16. A container device according to claim 15, wherein said testing device is further configured to direct a result provided by said testing device to said RFID device for retention therein.

17. A container device according to claim 14, wherein said testing device is configured to detect a substance selected from the group consisting of:
PCP;
amphetamines;
methamphetamines;
methadone;
benzodiazopenes;
barbiturates;
THC;
THC metabolites;
THCA;
cocaine;
cocaine metabolites;
ecstasy;
oxycodone;
propoxyphene;
methaqualone
opiates; and
combinations thereof.

18. A container device according to claim 14, further comprising a temperature device configured to determine a temperature of said specimen.

19. A container device according to claim 15, wherein said RFID device is non-removably engaged therewith.

20. A container device according to claim 16, further comprising a tamper-evident device operably engaged therewith.

21. A method for collecting and securing a chain of custody data set related to a specimen collected during specimen collection, data within said chain of custody data set being adapted to be input by a collector and said specimen being adapted to be provided by a donor, said method comprising the steps of:
guiding said collector, via a computer device, to determine said data within said chain of custody data set and to input complete data for said chain of custody data set into said computer device; and
directing said chain of custody data set to a printer operably engaged with said computer device, said printer configured to print said chain of custody data set as a bar code label configured to be capable of operably engaging a specimen container configured to receive said specimen.

22. A method according to claim 21, further comprising receiving data selected from the group consisting of: an electronic signature from said donor, an electronic signature from said collector, and combinations thereof.

23. A method according to claim 21, wherein said chain of custody data set is printed as a bar code label in a format selected from the group consisting of:
code 128;
PDF-417;
data matrix;
and combinations thereof.

24. A computer program product capable of directing the collection and securement of a chain of custody data set related to a specimen, data within said chain of custody data set being adapted to be input by a collector and said specimen being adapted to be provided by a donor, the computer program product comprising a computer-readable storage medium having computer-readable program code portions stored therein, the computer-readable program code portions comprising:
an executable portion for guiding said collector to determine data within said chain of custody data set and to input complete data for said chain of custody data set into a computer device; and
an executable portion for directing said chain of custody data set to a printer operably engaged with said computer device, said printer configured to print said chain of custody data set as a bar code label configured to be capable of operably engaging a specimen container configured to receive said specimen.

25. A computer program product according to claim 24, further comprising:
an executable portion for receiving data selected from the group consisting of: an electronic signature from said donor, an electronic signature from said collector, and combinations thereof.

26. A computer program product according to claim 24, wherein said chain of custody data set is printed as a two-dimensional (2D) bar code label.

27. A computer program product according to claim 24, wherein said chain of custody data set is printed as a bar code label in a format selected from the group consisting of:
code 128;
PDF-417;
data matrix;
and combinations thereof.
